(19) United States
(12) Patent Application Publication
(10) Pub. No.: US 2018/0161299 A1
(43) Pub. Date: Jun. 14, 2018

(54) ESSENTIAL NUTRIENTS AND RELATED METHODS

(71) Applicant: Balchem Corporation, New Hampton, NY (US)

(72) Inventor: Jonathan David Bortz, St. Louis, MO (US)

(21) Appl. No.: 15/834,953

(22) Filed: Dec. 7, 2017

Related U.S. Application Data
(60) Provisional application No. 62/432,468, filed on Dec. 9, 2016.

(51) Int. Cl. A61K 31/202; A61K 31/714; A61K 31/14; A61K 38/17

(52) U.S. Cl. CPC A61K 31/202; A61K 38/1709; A61K 31/14; A61K 31/714

(57) ABSTRACT

The present disclosure relates to pharmaceutical compositions for mammalian consumption containing essential fatty acids, protein, vitamins and choline, or combinations thereof, so constituted to optimize absorption and/or transport and/or efficiency of the co-ingredients of the composition. The pharmaceutical compositions are useful as supplements to provide nutrients to nutritionally deficient patients. The composition can contain essential fatty acids, such as EPA, DHA and/or ALA, cobalamin binding protein, such as Intrinsic Factor, vitamin B12 or a synthetic form thereof, choline, such as choline bitartrate, or combinations thereof.

### CROSS REFERENCE TO RELATED APPLICATIONS

This application claims the priority of U.S. provisional application No. 62/432,468, filed Dec. 9, 2016, which is hereby incorporated by reference in its entirety.

### FIELD OF THE INVENTION

The present disclosure relates to pharmaceutical compositions for mammalian consumption containing essential fatty acids, protein, vitamins, choline, or combinations thereof, so constituted to optimize absorption and/or transport and/or efficiency of the co-ingredients of the composition. The pharmaceutical compositions can be useful as supplements to provide nutrients to nutritionally deficient patients.

### BACKGROUND OF THE INVENTION

Good nutrition is essential for good mental and physical health. A person's nutritional status can affect the way they feel and how their body works. For example, nutrients are required to help persons grow and develop, repair body tissue, and build new muscle tissue. Poor nutrition, or malnutrition, can lead to nutritional deficiencies that can cause or exacerbate various disorders.

It is known that certain nutrients, such as essential fatty acids, choline, vitamins, and related proteins are important to various biological functions and pathways. Some of the biological functions and pathways are interrelated such that a deficiency in one or more of these nutrients can have negative effects on one or more of the various functions and pathways. Most persons who experience a deficiency in one or more of these nutrients suffer from a negative effect to one or more of their related biological functions or pathways.

The present disclosure relates to pharmaceutical compositions, and related methods, containing essential fatty acids, proteins, vitamins, choline, or combinations thereof that address and alleviate deficiencies among interrelated biological functions and pathways.

### SUMMARY OF THE INVENTION

The present disclosure relates to pharmaceutical compositions containing essential fatty acids, proteins, vitamins, choline, or combinations thereof.

In one embodiment, the present disclosure relates to a composition including an essential fatty acid selected from the group consisting of eicosapentaenoic acid (EPA), docosahexaenoic acid (DHA) and alpha-linolenic acid (ALA), and a cobalamin binding protein. For example, the composition can include in a single dosage form one or more essential fatty acids, such as DHA, with one or more cobalamin binding proteins, such as Intrinsic Factor or recombinant human Intrinsic Factor. The source of the essential fatty acids, such as DHA, can be animal, fish, plant, algae or microorganism produced. The source can also be a phosphatidylcholine containing DHA, a lysophosphatidylcholine containing DHA, or combination thereof, for example a TG or ethyl ester of a phospholid-DHA or a lysophospholipid-DHA. The amount of essential fatty acid present can be from about 10 mg to about 1500 mg and the amount of cobalamin binding protein present can be from about 10 μg to about 33,000 μg. The molar ratio of the essential fatty acid, e.g., DHA, to the cobalamin binding protein, e.g., Intrinsic Factor, can be from about 1:0.025 to about 1:0.00000005.

The composition can further include vitamin B12, choline, or both. In another embodiment, the present disclosure relates to a composition including an essential fatty acid selected from the group consisting of EPA, DHA and ALA, a cobalamin binding protein and vitamin B12. The amount of vitamin B12 present can be from about 1 to about 9,000 μg. The molar ratio of the cobalamin binding protein to the vitamin B12 can be from about 1:25,000 to about 1:0.00025.

In another embodiment, the present disclosure relates to a composition including an essential fatty acid selected from the group consisting of EPA, DHA and ALA, a cobalamin binding protein, vitamin B12 and choline. The amount of choline present can be from about 10 to about 5,000 mg. The molar ratio of choline to the essential fatty acid can be from about 1:0.0005 to about 1:50. The molar ratio of the vitamin B12 to choline can be from about 1:14 to about 1:200,000,000.

In one embodiment, the present disclosure relates to a composition including an essential fatty acid selected from the group consisting of EPA, DHA and ALA, a cobalamin binding protein, vitamin B12 and choline, wherein the molar ratio of essential fatty acid to cobalamin binding protein is about 1:0.025 to about 1:0.00000005, wherein the molar ratio of the cobalamin binding protein to the vitamin B12 is about 1:25,000 to about 1:0.00025, wherein the molar ratio of the vitamin B12 to choline is about 1:14 to about 1:200,000,000, and wherein the molar ratio of choline to the essential fatty acid is about 1:0.0005 to about 1:50.

In another embodiment, the present disclosure relates to a composition including an about 10 to about 1500 mg of an essential fatty acid selected from the group consisting of EPA, DHA and ALA, about 10 to about 33,000 μg of a cobalamin binding protein, about 0.25 to about 9,000 μg of vitamin B12, and about 10 to about 5,000 mg of choline, a biologically compatible salt thereof, or a phospholipid bound form and intermediates or derivatives thereof. In some embodiments, the essential fatty acid can be provided in one or more forms including as free fatty acids, triglycerides, methyl esters, ethyl esters, etc.

In another embodiment, the present disclosure relates to a method of providing supplemental nutrition to a patient, e.g. a mammal, including administering to the patient a composition as described herein containing essential fatty acids, protein, vitamins and choline, or combinations thereof.

In each embodiment of the present disclosure, the relative amounts of each component, the form as each component, or combinations thereof, can be provided to optimize the absorption, transport, cellular uptake, efficiency or combinations thereof of the other components of the composition. As a result, the composition and method can better treat or prevent the negative outcomes associated with nutritional deficiencies, such as those during pregnancy.

In other embodiments, the present disclosure relates to compositions, and related methods, including essential fatty acids (e.g., DHA), proteins (e.g., intrinsic factor) whereby the intrinsic factor can improve the absorption of ingested vitamin B12 to augment vitamin B12 levels which can prevent or mitigate the utilization and/or depletion of the body's choline levels, which in turn can support or improve the transport of DHA across biologic membranes (e.g., placenta in pregnant women, neuronal membranes in the central nervous system, etc.). For example, the compositions can be administered to pregnant women to prevent depletion of these essential nutrients and to benefit their offspring. The compositions can also be administered to treat patients, e.g., human subjects, with cognitive disorders (e.g., Alzheimer's dementia), neuropsychiatric disorders (e.g., schizophrenia, bipolar depression), to prevent or treat non-alcoholic fatty liver disease, metabolic resistance, metabolic syndrome as well as to reduce cardiovascular risk factors for cardiovascular disease.

The present disclosure relates to compositions, and related methods, including essential fatty acids (e.g., DHA), choline (e.g., choline bitartrate), vitamins (e.g., vitamin B12), related proteins (e.g., Intrinsic Factor), or combinations thereof to facilitate and optimize the efficient transfer of nutrients, such as essential fatty acids to the fetus of a pregnant patient. The compositions and related methods can also be used to replenish maternal levels of choline, essential fatty acids and vitamins.

One of the advantages of the present disclosure is the provision of two or more nutrients in specified amounts, forms and ratios to enhance or improve cellular uptake and function of the nutrients that play important roles in single carbon metabolism, fatty acid transport and cellular uptake, preservation of critical levels of choline for membrane integrity, fatty acid transport from the liver, protective effects on liver health as well as additional synergistic benefits between DHA and vitamin B12 for neuronal tissue. Each component of the composition can be present in an amount, form and/or ratio that enhances the functioning of the other components in the composition and in the body.

### BRIEF DESCRIPTION OF THE DRAWINGS

The foregoing and other features and advantages provided by the present disclosure will be more fully understood from the following description of exemplary embodiments when read together with the accompanying drawings, in which:

FIG. 1 shows triglyceride and phospholipid structures and the main components thereof. The triglyceride structure contains three fatty acids bound to a glycerol unit. The phospholipid structure contains two fatty acid groups bound to a glycerol unit, the glycerol unit bound to a phosphate group, and the phosphate group bound to a polar headgroup which can include choline.

FIG. 2 shows phospholipids and lyophospholipids in an aqueous solution forming monolayers, micelles and bilayer vesicles.

FIG. 3 shows an exemplary prenatal vitamin formulation in a gelatin capsule.

FIG. 4 shows an exemplary prenatal vitamin formulation in a gelatin capsule—tablet combination.

FIG. 5 shows an additional exemplary formulation in a gelatin capsule containing choline, Intrinsic Factor and DHA.

FIG. 6 shows an additional exemplary formulation in a gelatin capsule containing Intrinsic Factor and DHA.

FIG. 7 shows an additional exemplary formulation in a gelatin capsule containing choline, vitamin B12, Intrinsic Factor and DHA.

FIG. 8 shows an additional exemplary formulation in a gelatin capsule containing choline, vitamin B12, Intrinsic Factor and DHA.

### DETAILED DESCRIPTION OF THE INVENTION

The present disclosure relates to pharmaceutical compositions containing essential fatty acids, proteins, vitamins, choline, or combinations thereof.

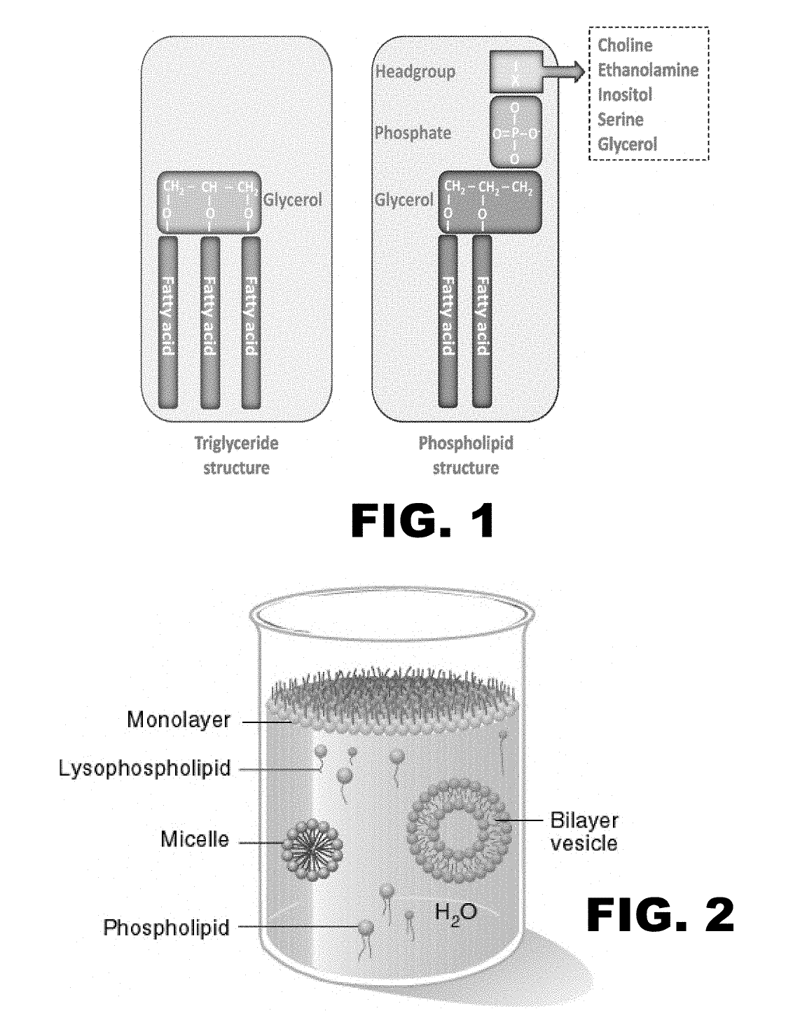

Essential fatty acids are fatty acids that humans and other animals must ingest because the body requires them for good health but cannot synthesize them. Fatty acids are typically found as either free fatty acids, triglycerides or phospholipids. FIG. 1 shows exemplary triglyceride and phospholipid structures. Triglycerides consist of three fatty acids esterified to a glycerol backbone. Phospholipids usually consist of two fatty acids esterified to a glycerol backbone, a phosphorous group and a headgroup. The headgroup confers a polarity on this molecule that give phospholipids the physical attributes of forming micelles (with the fat soluble fatty acids oriented inside these droplets and the water soluble headgroups oriented externally to interface with the external water environment. A common headgroup is choline and the resulting phospholipid is called phosphatidylcholine (PC).

Phosphatidylcholine can be synthesized by the phosphorylation (Kennedy) pathway via cytidine diphosphate (CDP-choline). PCs synthesized via the Kennedy pathway are capable of binding and transporting linoleic acid (18:2n-6) and oleic acid (18:1n-9). Phosphatidylcholine can also be synthesized by the phosphatidylethanolamine pathway (PEMT). PCs synthesized via the PEMT pathway are capable of binding and transporting DHA (22:6n-3) and ARA (20:4n-6). The transport of DHA across the placenta is particularly important because DHA is the one most required fatty acid for neurodevelopment in the developing fetal brain. DHA can constitute up to about 90% of the fatty acid content of the brain. Up to 7 g of DHA can be transported across the placenta in the 3^{rd} trimester via the PEMT pathway.

A substrate for the PEMT pathway is S-adenosyl methionine (SAM). SAM can be generated by adenosylation of methionine. Methionine is a product of methylation of homocysteine by either betaine (the oxidative product of choline) or methyl-cobalamin (a form of vitamin B12). For example, cobalamin can receive a methyl group from methyl-tetrahydrofolate which is incapable of methylating homocysteine directly, and does so indirectly by transferring its methyl group to cobalamin. The apparent redundancy of methylation of homocysteine to form methionine by both choline-betaine as well as methyl-cobalamin indicates its importance as a metabolic function. In instances when there is inadequate choline to supply betaine for homocysteine methylation, a portion of the burden is assumed by the folate-cobalamin route. Similarly, in instances when there is a folate or cobalamin deficiency, the burden is assumed by the choline route. This redundancy can have significant metabolic consequences when, for example, the supply of choline does not meet the physiologic requirements. When there is a choline deficiency, choline can be pulled from liver or muscle membranes, or diverted from the liver. This can cause manifestations of choline deficiency such as non-alcoholic fatty liver disease (NAFLD) and leakage of liver and muscle enzymes into the blood. Because the body can synthesize choline, choline is not considered a vitamin. Yet, the body generally cannot synthesize sufficient quantities of choline to meet the daily physiologic requirements and hence choline can be designated an essential nutrient.

In the presence of abundant or adequate choline supply, a folate/cobalamin deficit shifts the primary share of homocysteine methylation to the choline-betaine oxidative pathway. Inadequate intake of choline occurs globally in both developed and emerging countries. Nearly 90% of pregnant women do not meet their daily requirements of choline. The NHANES data generated for choline intake in the USA demonstrate that approximately 75% of the general population do not meet their daily recommended intake of choline. Similar intakes have been determined in a variety of other studies conducted in the European Union, China, Asia Pacific and New Zealand.

The global inadequacy of choline intake renders the PEMT pathway particularly vulnerable to restricting the synthesis of sufficient amounts of phosphatidylcholine for transport of DHA across the placenta. As a result, this vital mechanism for ensuring adequate amounts of DHA available for neurodevelopment of the fetus is particularly vulnerable to deficiencies of folate and cobalamin (vitamin B12). Following the successful programs to fortify food with folate, as well as the popularity of folate containing prenatal vitamins, the incidence of folate deficiency is quite rare and thought to be below 1%. Subclinical vitamin B12 deficiency, on the other hand, is very common with 39% of the US population between the ages of 26 and 83 have vitamin B12 deficiency (Tucker K L, et al; Am J Clin Nutr. 2000 February; 71(2):514-22). Subclinical vitamin B12 deficiency has been found to be as high as 75% in India, as a result of fact that the majority Hindi population eat no or very little animal source foods (which is the only source of vitamin B12 in the diet). Vitamin B12 deficiency stresses the already strained reserves of choline for DHA metabolism and other vital functions in which choline plays a key role.

In some embodiments, providing additional oral supplementation of vitamin B12 along with or in addition to a composition of essential fatty acids containing DHA is not the solution, in part, because Vitamin B12 is poorly absorbed (less than 1% in typical vitamin B12 supplementations). The primary reason for this is that vitamin B12 is a very large and unstable molecule weighing 1346 kilo Daltons and is preferably bound to a protein, e.g., Intrinsic Factor, to be preserved during its journey down the entire length of the small intestine to be recognized and absorbed by specific B12 receptors located in the distal ileum. These receptors (Cubilin) are responsible for the physiologic absorption of vitamin B12 and only recognize vitamin B12 bound to Intrinsic Factor.

Intrinsic Factor is a 399 amino acid protein produced by the parietal cells of the stomach and which binds to vitamin B12 in the duodenum to protect the molecule from proteolysis and hydrolysis and to deliver it to its receptor in the distal ileum. The production and supply of Intrinsic Factor can be compromised in certain autoimmune diseases like Pernicious Anemia as well as other conditions that contribute to alkalinizing the gastric fluid such as atrophic gastritis, chronic use of antacids, H₂-blockers or Proton Pump Inhibitors (PPIs). Hence, even having adequate intake of vitamin B12, in the absence of, or with inadequate Intrinsic Factor, there can be insufficient vitamin B12 absorbed via the physiologic absorption pathway leading to systemic and tissue vitamin B12 deficiency.

In some embodiments, the administration of bound vitamin B12, e.g., vitamin B12 complexed with Intrinsic Factor, can improve or ensure optimal physiologic absorption of vitamin B12. The administration of Intrinsic Factor alone can also provide a benefit by absorbing existing vitamin B12. For example, the administration of Intrinsic Factor in the unbound or apo form and not accompanied by supplemental vitamin B12 can improve the absorption of vitamin B12 contained in the dietary intake of a mammal as well as the amount of vitamin B12 that is recirculated via the entero-hepatic pathway. This is vitamin B12 that is excreted via the bile duct into the duodenum which can then be reabsorbed if there is enough Intrinsic Factor. Hence an enhancement of serum vitamin B12 could be seen even in the total absence of dietary vitamin B12—whether from the diet or in form of an oral supplement.

As used herein, the term “patient” comprises any and all organisms and includes the term “subject,” “mammal,” “human,” “mother” and “baby.”

As used herein, the term “administering” refers to the act of giving a composition to a patient or otherwise making such composition available to a patient.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments.

The compositions of the present disclosure can be any solid, oral dosage form in which the dose is to be administered to the patient or subject. The composition can be in for form of a tablet, capsule, pill or film. In one embodiment, the composition can be formulated as a daily supplement for humans. In another embodiment, the composition can be formulated as a preconception, prenatal or peripartum supplement.

In one embodiment, the present disclosure relates to a composition including an essential fatty acid, such as one selected from the group consisting of EPA, DHA and ALA, and a cobalamin binding protein. For example, the composition can include about 10 to about 1500 mg of an essential fatty acid selected from the group consisting of EPA, DHA and ALA, and about 10 to about 33,000 μg of a cobalamin binding protein.

### Essential Fatty Acids

As provided herein, the term “essential fatty acids” includes eicosapentaenoic acid (EPA), docosahexaenoic acid (DHA) and alpha-linolenic acid (ALA). As such, the essential fatty acids can be selected from the group consisting of EPA, DHA, ALA or combinations thereof. The fatty acids can be obtained in solid form, such as in a whole-cell microbial product, or in liquid form, such as an oil. The source of essential fatty acids, e.g., DHA, can be from one or more of animal, fish, plants, algae or microorganism production. In one embodiment, the source of the fatty acids, e.g., DHA, is from algae oil.

The human brain is comprised of approximately 60% fatty acids, 90% of which is DHA. Peak brain growth in most animals, such as humans, occurs in the third trimester of pregnancy. The composition of the present disclosure can be administered during the third trimester of pregnancy. During the last stages of pregnancy, in humans, approximately 7 grams per day of fatty acids are transferred across the placenta. The composition of the present disclosure can provide for the transfer (to the brain, across the placenta, or both) of sufficient amounts of fatty acids, choline, or both per day, e.g., about 0.1, 0.2, 0.3, 0.4, 0.5, 0.6, 0.7, 0.8, 0.9, 1, 2, 3, 4, 5, 6, 7, 8, 9, 10 or 15 grams per day. These values can be used to define a range, such as about 1 to about 10 grams per day. The fatty acids transferred across the placenta primary consist of DHA, which can cause depleted maternal levels of DHA. When administered to the mother, the composition of the present disclosure can restore the depleted material levels of nutrients, e.g., DHA. Breast milk fat is also rich in DHA. The transport of DHA is favored over other long chain fatty acids, e.g., EPA, by the both the placenta and breast.

Omega-3 fatty acids can also affect cognitive decline and dementia. DHA and EPA can increase brain volume and lower degrees of white matter hyperintensities. Red blood cell DHA and EPA concentrations can provide higher total brain and hippocampal volumes. Higher relative concentrations of plasma EPA can provide a reduced brain atrophy rate in the medial temporal lobe. DHA, an omega-3 fatty acid highly concentrated in the brain and the outer segments of retinal rods and cones, constitutes around 50% of the total polyunsaturated fatty acids. DHA participates in a number of neuronal processes including neurogenesis, neuroplasticity, neuron differentiation and survival, membrane integrity and fluidity. Maternal supplementation of DHA during gestation can provide neuroprotective effects against prenatal stress-induced brain dysfunction, hyperoxic injury and hypoxic ischemic injury.

The amount of essential fatty acids in the composition can be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or about 99 wt % of the composition. These values can also be used to define a range, such as from about 10 to about 90 wt %, or about 40 to about 80 wt %.

The amount of essential fatty acids in the composition can be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1050, 1100, 1150, 1200, 1250, 1300, 1350, 1400, 1450, 1500, 1550, 1600, 1650, 1700, 1750, 1800, 1850, 1900, 1950, 2000, 2100, 2200, 2300, 2400, 2500, 2600, 2700, 2800, 2900, 3000, 3500, 4000, 4500 and about 5000 mg. These values can also be used to define a range, such as from about 10 to about 1500 mg, or about 300 to about 700 mg.

Phosphatidylcholines are a class of phospholipids that incorporate fatty acids and choline. They are a major component of biological membranes. They are also a member of the lecithin group of yellow-brownish fatty substances occurring in animal and plant tissues. Phosphatidylcholine is a major component of lecithin. The transport of phosphatidylcholines can also be assisted by phosphatidylcholine transfer protein. In some embodiments, the composition can contain a sufficient amount of phosphatidylcholine transfer protein, such as in an isolated and purified form, to assist in phosphatidylcholine, fatty acids and/or choline transport.

The phospholipid headgroups are characteristically hydrophilic and when they are connected to a glycerol backbone containing the hydrophobic fatty acids by a phosphate bridge, they transform this phospholipid complex into a polar lipid. This confers unique properties with respect to water and oil, with the polar heads oriented toward water and the fatty acid tails toward oil. When suspended in water, the phospholipids can spontaneously rearrange into a variety of ordered structures depending on the physiologic demands. FIG. 2 shows some of the various structures. In the case of a water-oil interface, the phospholipids can assemble as a monolayer with the hydrophobic fatty acid end pointing away from water and when immersed in water, this fatty acid end will turn inward and form vesicles or spheres.

The amphipathic phospholipids play an important in their intact state which constitutes four different molecules. They can also be assembled and dissembled under the influence of various hydrolytic enzymes both in the gastrointestinal tract and the systemic circulation. Four phospholipases can play a role in purposing and repurposing these complexes for various signaling and functional activities throughout the body. The phospholipases are PLA₁, PLA₂, PLC and PLD. These hydrolases can act at various sites on the phospholipid and under different stimuli and in different biologic locations. For example, PLA₂ can hydrolyze the bond between the fatty acid in the second position (sn-2 or middle carbon of the glycerol backbone). PLA₁ can hydrolyze the fatty acid off the backbone at position 1 (sn-1 or carbon furthest from the phosphate bridge which is attached to the sn-3 position of the carbon chain). When one fatty acid is removed, a lysophospholipid is formed.

Plasma fatty acids, e.g., DHA, can be carried in two forms, as non-esterified fatty acids which supply fatty acids, e.g., DHA, to platelets, and as lysophosphatidylcholine (LPC) which delivers fatty acids, e.g., DHA, to red blood cells. There can be a preferential uptake of fatty acid, e.g., DHA, from a lysophospholipid, e.g., LPC-DHA, compared with non-esterified fatty acids. The composition of the present disclosure can provide adequate choline to promote the assembly and generation of PC-essential fatty acids, e.g., PC-DHA, and lysoPC-essential fatty acids, e.g., LPC-DHA, which can provide essential fatty acids, e.g., DHA, choline or both, for transfer and to neural and other tissues.

Regarding transfer, such as across the placenta, pregnancy increases the demands for PC as produced by the PEMT pathway. PEMT-PC can be selectively transferred to the fetus. The PEMT-PC containing essential fatty acids, e.g., DHA, can be incorporated into VLDL and made available to the placenta for transfer into the fetus. The demands for PC production via the CDP pathway in pregnancy also relates to the enhanced hepatic lipid load experienced by mothers in late pregnancy. PC can be used to synthesize VLDL which can facilitate transport of triglycerides out of the liver. Pregnancy also increases the demands for choline, especially in the third trimester. Choline is driven down both the phosphorylative and oxidative pathways and when increasing the choline intake in pregnant women from 480 mg to 930 mg there is little, if any, choline excreted in the urine, highlighting a huge capacity for metabolizing an amount in excess of currently recommended dietary intakes and underlining a substantial increase in the requirements of these nutrients during and associated with pregnancy. The composition of the present disclosure can be used to meet the demands for phosphatidylcholines, choline, essential fatty acids, vitamins and other nutrients. The availability of sufficient amounts of these nutrients, such as phosphatidylcholines and choline, is associated with efficient transport of essential fatty acids, e.g., DHA, across the placenta and into neurologic tissue to ensuring adequate and/or optimal essential fatty acid, e.g., DHA, concentrations in the brain of the developing fetus.

The essential fatty acids, choline, or both in the composition can be in the form of one or more phosphatidylcholines, lysophosphatidylcholines or combinations thereof, e.g., a phospholipid bound form and intermediates or derivatives thereof. The percentage of essential fatty acids, e.g., EPA, DHA, ALA, or any one particular essential fatty acid in the composition in the form of one or more phosphatidylcholines, lysophosphatidylcholines or combinations thereof, can be about, more than about, or less than about, 0%, 1, 5, 10, 15, 20, 25, 30, 35, 40, 45, 50, 55, 60, 65, 70, 75, 80, 85, 90, 95, 99 or about 100%. These values can be used to define a range, such as about 20% to about 80%. In some embodiments, the percentage of essential fatty acids and choline in the composition in the form of one or more phosphatidylcholines, lysophosphatidylcholines or combinations thereof is about equal, e.g., the majority of both are in the form of one or more phosphatidylcholines, lysophosphatidylcholines or combinations thereof. In some embodiments, the source of the PC-DHA can be omega-3 fatty acid enriched eggs or Krill oil.

The fatty acids contained in the phosphatidylcholines, lysophosphatidylcholines or combinations thereof can be about 5, 10, 15, 20, 25, 30, 35, 40, 45, 50, 55, 60, 65, 70, 75, 80, 85, 90, 95, 98, 99 or about 100% EPA. These values can be used to define a range such as between about 50 and about 99%.

The fatty acids contained in the phosphatidylcholines, lysophosphatidylcholines or combinations thereof can be about 5, 10, 15, 20, 25, 30, 35, 40, 45, 50, 55, 60, 65, 70, 75, 80, 85, 90, 95, 98, 99 or about 100% DHA. These values can be used to define a range such as between about 50 and about 99%.

The fatty acids contained in the phosphatidylcholines, lysophosphatidylcholines or combinations thereof can be about 5, 10, 15, 20, 25, 30, 35, 40, 45, 50, 55, 60, 65, 70, 75, 80, 85, 90, 95, 98, 99 or about 100% ALA. These values can be used to define a range such as between about 50 and about 99%.

### Cobalamin Binding Protein

Vitamin B12 is an important component of the essential fatty acid and choline related biological functioning and pathways, as described herein. Vitamin B12 is absorbed from the gastro-intestinal tract via specific B12 receptors located in the distal small intestine. Vitamin B12 is a very large molecule (about 1300 Daltons) and is susceptible to hydrolysis and enzymatic degradation as it passes through the GI tract to the distal ileal B12 receptors. A cobalamin binding protein (CBP) can be used to bind the vitamin B12 to protect it against degradation. Examples of cobalamin binding proteins include recombinant human Intrinsic Factor (rh-IF), haptocorrin, and transcobalamin, see U.S. patent application Ser. No. 12/357,622, the content of which is incorporated herein by reference in its entirety. Intrinsic Factor may be expressed and purified via standard methodology. In a specific embodiment, Intrinsic Factor may be expressed and purified from a transgenic plant, such as Arabidopsis. The expressed and purified IF may be from any species. Non-limiting examples include human, mouse, rat, dog, cat, cattle, pig, non-human primates, and horse.

The cobalamin binding protein, e.g., Intrinsic Factor, can have a very high affinity for vitamin B12. Once bound, the complex can afford protection for vitamin B12 against degradation. The complex can be recognized by the vitamin B12 receptor (e.g., Cubilin) which then transfers the vitamin B12 across the small intestinal cell lining into the circulation. The body uses this receptor-mediated pathway to absorb a required amount of vitamin B12. The cells of the body can utilize up to about 5.2 μg/day of vitamin B12.

When large oral doses of vitamin B12 are ingested orally (e.g., 500 μg to 5000 μg) less than about 1% of vitamin B12 is absorbed by diffusion from the gastrointestinal tract. The unabsorbed vitamin B12 can be broken down into vitamin B12 fragments, called analogues in the GI tract, that can also be absorbed into the circulation. Some vitamin B12 analogues can inhibit vitamin B12 dependent metabolic processes. For example, it has been shown that some Alzheimer's Disease patients have significantly higher vitamin B12 analogue levels that may be contributing to a functional B12 deficiency and some of the manifestations of Alzheimer's. Large oral and systemic doses of B12 can generate a more than 10-fold increase in the amount of B12 analogues present in the GI tract and in the circulation. Hence, a very circumscribed amount of vitamin B12 can be administered to meet cellular metabolic requirements, as opposed to massive doses of vitamin B12, to avoid the formation of and the negative effects associated with vitamin B12 analogues. The composition of the present disclosure can provide the required amount of cobalamin binding protein (e.g., Intrinsic Factor) to facilitate physiologic receptor mediated absorption of the right amount of vitamin B12 to ensure cellular B12 adequacy but not B12 and B12 analogue excess with its deleterious effects. The composition of the present disclosure can also include specific amounts of vitamin B12, for oral administration, alone or in combination with essential fatty acids, choline, a cobalamin binding protein (e.g., Intrinsic Factor) or combinations thereof.

The composition of the present disclosure can reduce the formation of analogues of vitamin B12 and improve or ameliorate conditions associated therewith. Specific amounts of vitamin B12 can be bound, un-bound, or combinations thereof to the cobalamin binding protein (e.g., Intrinsic Factor). In some embodiments of the present disclosure, the composition can deliver a physiologic dose of vitamin B12 as opposed to a mega dose, and avoid generating vitamin B12 analogues that can interfere with vitamin B12's metabolic functions.

The amount of cobalamin binding protein (e.g., Intrinsic Factor) in the composition can be about 0.00001, 0.00002, 0.00005, 0.0001, 0.0002, 0.0005, 0.001, 0.002, 0.003, 0.004, 0.005, 0.006, 0.007, 0.008, 0.009, 0.01, 0.015, 0.02, 0.025, 0.03, 0.035, 0.04, 0.045, 0.05, 0.055, 0.06, 0.065, 0.07, 0.075, 0.0.8, 0.085, 0.09, 0.095, 0.1, 0.2, 0.3, 0.4, 0.5, 0.6, 0.7, 0.8, 0.9, 1, 2, 3, 4, 5, 6, 7, 8, 9 or about 10 wt %. These values can also be used to define a range, such as from about 0.001 to about 3.3 wt %.

The amount of cobalamin binding protein (e.g., Intrinsic Factor) in the composition can be about 1, 2, 3, 4, 5, 6, 7, 8, 9, 10, 15, 20, 30, 40, 50, 60, 70, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, 1000, 1100, 1150, 1200, 1250, 1300, 1350, 1400, 1450, 1500, 1550, 1600, 1650, 1700, 1750, 1800, 1850, 1900, 1950, 2000, 2150, 2200, 2250, 2300, 2350, 2400, 2450, 2500, 2550, 2600, 2650, 2700, 2750, 2800, 2850, 2900, 2950, 3000, 3150, 3200, 3250, 3300, 3350, 3400, 3450, 3500, 3550, 3600, 3650, 3700, 3750, 3800, 3850, 3900, 3950, 4000, 4150, 4200, 4250, 4300, 4350, 4400, 4450, 4500, 4550, 4600, 4650, 4700, 4750, 4800, 4850, 4900, 4950, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, 15000, 20000, 25000, 30000, 33000, 35000 or about 50000 μg. These values can also be used to define a range, such as from about 10 and about 33,000 μg, or from about 50 to about 400 μg. The molar ratio of essential fatty acid to cobalamin binding protein (e.g., Intrinsic Factor) in the composition can be about, more than about, or less than about 1:1; 1:0.5; 1:0.2; 1:0.1; 1:0.05; 1:0.025; 1:0.02; 1:0.01; 1:0.005; 1:0.002; 1:0.001; 1:0.0005; 1:0.0002; 1:0.0001; 1:0.00005; 1:0.00002; 1:0.00001; 1:0.000005; 1:0.000002; 1:0.000001; 1:0.0000005; 1:0.0000002; 1:0.0000001; 1:0.00000005; 1:0.00000002; 1:0.00000001; 1:0.000000005; 1:0.000000002 or to about 1:0.00000001. These values can also be used to define a range, such as from about 1:0.025 to about 1:0.00000005.

The proteins can be administered alone, e.g., in the apo-form, or in conjunction with cobalamin or a cobalamin analog, e.g., in the holo-form. A “holo-form” of a cobalamin binding protein is a complex of the cobalamin binding protein and its ligand, e.g., cobalamin or a cobalamin analog or synthetic form. An “apo-form” of a cobalamin binding protein is the cobalamin binding protein which is ready to bind a cobalamin or a cobalamin analog or synthetic form.

The purity of the cobalamin binding protein (e.g., Intrinsic Factor) in the composition can vary. In some embodiments, the cobalamin binding protein can be highly purified. For example, the cobalamin binding protein can have a purity value about, or more than about 50, 55, 60, 65, 70, 75, 80, 85, 90, 95, 98 or about 99 wt %. In other embodiments, the cobalamin binding protein can be less purified. For example, the cobalamin binding protein can have a purity value about, or less than about 1, 5, 10, 15, 20, 25, 30, 35, 40, 45 or about 50 wt %. The cobalamin binding protein in the composition can be unpurified from an animal source or rh-IF expressed in plant material and in powdered or liquid form.

In some embodiments, the composition can include other components that exhibit intrinsic factor activity, such as dessicated stomach substance (DSS). DSS is a crude extract from animals that has some weak intrinsic factor activity.

### Vitamin B12

As used herein, the term “vitamin B12” or “B12” can include vitamin B12 or synthetic forms of vitamin B12.

The present disclosure relates to a composition including an essential fatty acid selected from the group consisting of EPA, DHA and ALA, a cobalamin binding protein and vitamin B12. The present disclosure also relates to a composition including an essential fatty acid selected from the group consisting of EPA, DHA and ALA, and vitamin B12. The present disclosure also relates to a composition including a cobalamin binding protein and vitamin B12.

Vitamin B12, or cobalamin, is a water-soluble vitamin that has a key role in the normal functioning of every cell in the body and also functional integrity of the brain and nervous system. It is one of eight B vitamins. It is involved in the metabolism of every cell of the human body, and can affect fatty acid metabolism. A common form of the vitamin is cyanocobalamin, produced by chemically modifying bacterial hydroxocobalamin. In the body, it is converted into the human physiological forms methylcobalamin and 5′-deoxyadenosylcobalamin. In this process small amounts of cyanide ion is produced. Cyanide-free synthetic forms of the vitamin include hydroxocobalamin, methylcobalamin, and adenosylcobalamin. The composition can include Vitamin B12, synthetic forms thereof, or combinations thereof. The form of vitamin B12 can be selected from the group consisting of cyanocobalamin, methylcobalamin, hydroxocobalamin, adenosylcobalamin and combinations thereof. Regardless of what form of vitamin B12 is used, it can be complexed with a cobalamin binding protein.

Vitamin B12 is involved in the methylation of homocysteine to methionine, which is the key substrate for generation of S-adenosyl methionine (SAM) which is the main methyl donor in single carbon metabolism. SAM is also the main substrate for the PEMT (phosphotidyl ethanolamine methyl transferase) pathway that, under the influence of estrogen, can generate phosphotidyl choline that can form phospholipids with DHA and ARA, which can then be transferred across the placenta into the developing fetus. Hence, vitamin B12 is also involved in the delivery and transfer of omega-3 fatty acids, the benefit of which includes neurological tissue and brain development in the fetus. The Folate/B12 pathway and Choline/Betaine pathway are each available for single carbon metabolism. These pathways have some redundancy to ensure that when a component or substrate in one pathway is deficient, the other pathway can ensure sufficient methylation is adequately maintained, such as for gene transcription. The Folate/B12 pathway and Choline/Betaine pathway can each be affected by seasonal or other changes in availability of any of these components or substrates.

S-adenosylmethionine is the major methyl donor in the cell and is produced from methionine by L-methionine S-adenosyltransferase (MAT). SAM is involved in numerous cellular reactions, including DNA methylation and synthesis of phosphatidylcholine, as well as in reactions involving neurotransmitters, creatine, carnitine, and antioxidants (such as glutathione and taurine). Methionine can be supplied either by the diet or can be generated from homocysteine via methionine synthase or via betaine homocysteine methyltransferase (BHMT). The BHMT pathway is particularly active in the liver and the kidney, which are the main organs that store large amounts of betaine.

Methionine, betaine, choline, and 5-methyltetrahydrofolate (5-MTHF) are important dietary sources of labile methyl groups in mammalian cells. 5-MTHF cannot donate its methyl group directly to homocysteine and is required to transfer it to cobalamin (vitamin B12). Three enzymes are required to produce methyl groups via the folate cycle: serine hydroxymethyl transferase (SHMT), MTHFR, and methionine synthase. Methionine synthase, a vitamin B12-dependent enzyme, facilitates the last step of methyl group transfer from 5-MTHF to homocysteine to form methionine.

Furthermore, in order to be able to recycle 5-MTHF efficiently to tetrahydrofolate to replenish folate levels to perform many other metabolic functions, adequate amounts of the methylated vitamin B12 (methylcobalamin) have to be available. If not, 5-MTHF can become trapped and can cause a functional folate deficiency because folate may not be replenished via this methylated vitamin B12 pathway. The success of the Folate methylation of homocysteine can depend on vitamin B12 levels and in view of the folate fortification programs in many countries and the ubiquitous presence of folate in prenatal vitamins and multivitamins, where folate deficiency is a rarity (<1% prevalence), it is B12 deficiency (approximately 39% in the US, and much higher in countries with lower animal food source diets), an adequate vitamin B12 level is the rate limiting step in the folate pathway. Folate deficiency can be partly attenuated when choline is available and vice versa. For example, choline and phosphatidylcholine can be depleted in the livers of rats fed a folate-deficient diet. In contrast, consumption of a choline-deficient diet can decrease hepatic folate stores. A choline-deficient diet can lower methionine formation in animal livers by 20%-25% because less choline is available for conversion into betaine. A choline-deficient diet can increase SAM utilization in the liver, to convert phosphatidyl-ethanolamine into phosphatidylcholine via PEMT.

The composition of the present disclosure can provide sufficient choline intake in patients suffering from a folate deficiency to reduce or ameliorate the folate deficiency, or the effects thereof. Folate deficiency can result in increased hepatic betaine:homocysteine methyltransferase activity and decreased hepatic betaine and choline. Increased hepatic betaine:homocysteine methyltransferase activity found in a patient with reduced 5, 10-methylenetetrahydrofolate reductase activity can indicate that choline-based methylation is upregulated also during methylfolate deficiency. The interdependence of Folate/B12 and Choline/betaine pathways has a functional purpose to ensure that when availability of one methyl donor/carrier participant is restricted, the other system becomes more engaged and therefore adequacy in both systems has a substrate sparing coeffect.

Both vitamin B12 and essential fatty acids play a role in maintaining form, function and development of neuronal and neurologic tissue. Deficiencies of either one are associated with neuropsychiatric and developmental manifestations. The composition of the present disclosure addresses these deficiencies.

Homocysteine is a nonessential, sulfur-containing amino acid synthesized endogenously from methionine. Raised plasma total homocysteine (tHcy) is a modifiable risk factor for cognitive impairment, dementia, and AD. The atrophy rate of the brain can be faster at low plasma vitamin B12 concentrations and at high plasma tHcy concentrations. The treatment of older persons with high doses of homocysteine-lowering B vitamins can reduce the global brain atrophy rate, as well as atrophy rates in those gray matter regions most commonly associated with AD. The composition of the present disclosure can also address these treatments.

One of the key biomarkers of vitamin B12 deficiency is an elevation of homocysteine. Homocysteine is a powerful generator of reactive oxygen species which induce neuronal DNA damage, triggering apoptosis and affects synaptic and glial function. The brain is highly susceptible to oxidative cellular damage due to high metabolic load and poor antioxidant defense system. Furthermore, omega-3 fatty acids are susceptible to degradation due to increased oxidative stress. In view of the fact that neurologic tissue contains such a high percentage of DHA, this tissue is particularly vulnerable to oxidative damage induced by iincreased plasma homocysteine levels in offspring which can occur as a consequence of maternal vitamin B12 deficiency. Further, reduced plasma levels of vitamin B12 and DHA and increased homocysteine levels can be present in schizophrenic patients and suggests a role in the psychological abnormality underlying the disease. A negative association between maternal plasma homocysteine and erythrocyte DHA levels in pregnancy complications, e.g., preeclampsia, exist. Hyperhomocysteinemia through the mediation of oxidative stress can produce changes in structure and function of cerebral blood vessels. High levels of homocysteine can cause damage and leakage to hippocampal microvasculature leading to vascular remodeling which can disrupt the blood-brain barrier. Homocysteine can also inhibit angiogenesis through the inhibition of vascular endothelial growth factor (VEGF). The composition of the present disclosure can include essential fatty acids and vitamin B12 which can reduce or prevent the presence or effects of homocysteine. The composition can protect mother and baby, and can improve methylation and assembly of phospholipids.

The composition of the present disclosure can also improve or maintain the equilibrium between neurotrophic and neurotoxic factors in the central nervous system. Neurotrophins are growth factors that influence the proliferation, differentiation, survival and death of neuronal and non-neuronal cells. Reduced levels of neurotrophins, such as NGF (Nerve Growth Factor) and BDNF (Brain Derived Neurotrophic Factor), in the brain can be caused by vitamin B12 deficiency. Reduced levels of BDNF can affect the pathophysiology of various psychiatric disorders like schizophrenia, Alzheimer's disease, Parkinson's disease, and Huntington's disease. Lower serum NGF levels in and BDNF levels in the schizophrenic patients can be associated with cognitive impairment. High neurotrophin expression in the brain can act as neuroprotective against neurological diseases.

Omega-3 fatty acids can act as neuroprotective agents against neurological insults through the BDNF signaling pathways. DHA supplementation in aged mice can improve cognitive dysfunction through increased BDNF levels. DHA can increase neurotrophins in the brain by increasing membrane fluidity, reducing oxidative stress, through neuroprotection. Combined supplementation of both vitamin B12 and omega-3 fatty acids together can increase the levels of BDNF in the cortex and hippocampus region of the brain. Altered neurotrophins and their downstream signaling pathway affected by the deficiency of vitamin B12 and omega-3 fatty acids can be reduced or prevented by the composition of the present disclosure. The composition of the present disclosure can provide an adequate or sufficient amount of vitamin B12 as a prerequisite for optimizing the effect of essential fatty acids, choline, or both in the body, such as upon brain tissue integrity and signaling.

The dysregulation of the one-carbon metabolism can result in brain disorders like schizophrenia, bipolar disorder, autism and depression. Vitamin B12 is important cofactor in one carbon cycle and is involved in the formation of S-adenosyl methionine (SAM). SAM is a universal methyl donor for important methylation reactions including methylation of DNA, neurotransmitters and phospholipids. Phospholipids utilize methyl groups for the conversion of phosphatidylethanolamine (PE) to phosphatidylcholine (PC). The conversion of PE to PC in biological membranes can affect mobilization of DHA from liver to plasma and brain. Patients of Alzheimer's disease can have high levels of circulating homocysteine and decreased mobilization of DHA from the liver into plasma and peripheral tissues which can contribute to cerebrovascular and neurodegenerative changes. The one-carbon metabolism can influence epigenetic modifications which in turn can produce long-term changes in the brain affecting memory, learning, cognition and behavior. Epigenetics can induce changes in the chromatin without disrupting the basic DNA sequence. DNA methylation is the most widely studied form of epigenetic modification which occurs through one-carbon metabolism. DNA methylation/demethylation can play an important role in learning and memory as suppression of DNA methylation can impaired long term potentiation. Epigenetic modifiers can play a role in neurodevelopment. An association between memory and changes in DNA methylation in the BDNF gene can exist. DNA methylation can also control BDNF expression during development of the forebrain in mice.

An adequate supply of nutrients which can be a source of methyl groups to the brain can promote proper functioning. The composition of the present disclosure, such as a composition including Vitamin B12, essential fatty acids, or both, can be a modifier of epigenetics and affect the one-carbon cycle. The compositions can reduce or prevent the occurrence of altered global methylation patterns in the brain of an offspring as a consequence of imbalanced (e.g., excess folate and vitamin B12 deficient) maternal micronutrients in the mother.

The amount of vitamin B12 or a synthetic form of vitamin B12 in the composition can be about 0.00001, 0.00002, 0.00005, 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.0.8, 0.09, 0.1, 0.2, 0.3, 0.4, 0.5, 0.6, 0.7, 0.8, 0.9, 1, 2, 3, 4, 5, 6, 7, 8, 9 or about 10 wt %. These values can also be used to define a range, such as from about 0.0001 to about 0.1 wt %.

The amount of vitamin B12 or a synthetic form of vitamin B12 in the composition can be about 0.05, 0.1, 0.2, 0.25, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 105, 110, 115, 120, 125, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1050, 1100, 1150, 1200, 1250, 1300, 1350, 1400, 1450, 1500, 1550, 1600, 1650, 1700, 1750, 1800, 1850, 1900, 1950, 2000, 2100, 2200, 2300, 2400, 2500, 2600, 2700, 2800, 2900, 3000, 3500, 4000, 4500, 5000, 5500, 6000, 6500, 7000, 7500, 8000, 8500, 8800, 9000, 9500 and about 10000 μg. These values can also be used to define a range, such as from about 0.25 to about 8800 μg, from about 1 to about 9000 μg, from about 1 to about 5000 μg, from about 1 to about 5 μg, from about 2.5 to about 10 μg, from about 5 to about 25 μg, from about 20 to about 50 μg, or from about 40 to about 100 μg.

The molar ratio of cobalamin binding protein (e.g., Intrinsic Factor) to vitamin B12, in the composition can be about, more than about, or less than about 1:100,000; 1:50,000; 1:25,000; 1:20,000; 1:10,000; 1:5,000; 1:2,000; 1:1000; 1:500; 1:200; 1:100; 1:50; 1:20; 1:10; 1:5; 1:2; 1:1; 1:0.5; 1:0.2; 1:0.1; 1:0.05; 1:0.02; 1:0.01; 1:0.005; 1:0.002; 1:0.001; 1:0.0005; 1:0.00025; 1:0.0002; 1:0.0001; 1:0.00005; 1:0.00002; to about 1:0.00001. These values can also be used to define a range, such as from about 1:25,000 to about 1:0.00025.

### Choline

In one embodiment, the present disclosure relates to a composition including an essential fatty acid selected from the group consisting of EPA, DHA and ALA, a cobalamin binding protein, vitamin B12 and choline, one or both being a biologically compatible salt thereof, or a phospholipid bound form and intermediates or derivatives thereof.

In another embodiment, the present disclosure relates to a composition including an essential fatty acid selected from the group consisting of EPA, DHA and ALA, and choline, one or both being a biologically compatible salt thereof, or a phospholipid bound form and intermediates or derivatives thereof.

Choline is a water-soluble essential nutrient. Choline generally refers to the various quaternary ammonium salts containing the N,N,N-trimethylethanolammonium cation, below, wherein the X denotes a counteranion.

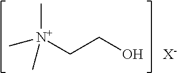

Choline is not strictly speaking classified as a vitamin because the body can synthesize some choline. It is recognized as an essential nutrient because the body cannot synthesize adequate amounts sufficient to meet all of the related metabolic requirements for choline. The daily requirements for choline are dramatically increased in the third trimester as peak brain and central nervous system development occurs. Choline is also present in high concentrations in breast milk, as neurologic and brain development continue postpartum at an accelerated rate for approximately 2 to 3 years postpartum. Choline is an important component of phospholipids in the cell membranes. Choline is phosphorylated by choline kinases (CHK) to phosphorylcholine within cells, and, after several biosynthetic processes, finally is integrated into phospholipids.

Choline, as used herein, refers not only to the isolated choline molecule (i.e., free choline), but also to any biologically compatible salt of choline (e.g., choline bitartrate) or and complex compound, chelate, prodrug, etc. containing choline wherein choline can be released or produced by degradation or other means from the complex compound, chelate, prodrug, etc. In certain embodiments, the choline form conforms to the USP monograph. The choline can be included in the composition in one or more of the forms selected from, but not limited to, choline bitartrate, choline chloride, choline hydroxide, choline dihydrogen citrate, choline salicylate, choline magnesium trisalicylate, choline carbonate, as well as other forms as described herein. In one embodiment, the choline salt is choline chloride. In another embodiment, the choline salt is choline bitartrate. In yet another embodiment, the choline salt is choline dihydrogen citrate. The choline compositions can also contain a blend of one or more choline salts or forms, such as a mixture of choline chloride and choline bitartrate.

The choline salt can also be optically active to polarizing light. The choline salt can have an excess of one or more optical isomers. For example, the choline salt can be the L(+) isomer of tartrate. The optically active light can be rotated by differing degrees. In one embodiment, optically active light is rotated more than about +17.5 degrees to polarizing light. In another embodiment, optically active light is rotated less than about +17.5 degrees to polarizing light.

In some embodiments, the choline salt is optically inactive to polarizing light. The choline salt can be used as a racemic mixture. For example, the choline salt can be a racemic mixture of the D and L isomers of tartrate. In some embodiments, the choline salt is in the natural form of tartrate.

The amount of choline, choline salt or choline salts in the composition can be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or about 99 wt %. These values can also be used to define a range, such as from about 5 to about 50 wt %, or about 10 to about 25 wt %.

The choline cation concentration in the composition can be greater than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or about 99 wt %. These values can also be used to define a range, such as from about 5 to about 50 wt %, or about 10 to about 25 wt %.

The amount of choline, choline salt or choline salts in the composition can be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1050, 1100, 1150, 1200, 1250, 1300, 1350, 1400, 1450, 1500, 1550, 1600, 1650, 1700, 1750, 1800, 1850, 1900, 1950, 2000, 2100, 2200, 2300, 2400, 2500, 2600, 2700, 2800, 2900, 3000, 3500, 4000, 4500 and about 5000 mg. These values can also be used to define a range, such as from about 10 to about 5000 mg, about 25 to about 200 mg, or about 400 to about 600 mg, or about 400 to about 450 mg, or about 500 to about 600 mg.

The molar ratio of choline to the essential fatty acid in the composition can be about, more than about, or less than about 1:0.0001; 1:0.0002; 1:0.0003; 1:0.0004; 1:0.0005; 1:0.0006; 1:0.0007; 1:0.0008; 1:0.0009; 1:0.001; 1:0.002; 1:0.003; 1:0.004; 1:0.005; 1:0.006; 1:0.007; 1:0.008; 1:0.009; 1:0.01; 1:0.02; 1:0.03; 1:0.04; 1:0.05; 1:0.06; 1:0.07; 1:0.08; 1:0.09; 1:0.1; 1:0.2; 1:0.3; 1:0.4; 1:0.5; 1:0.6; 1:0.7; 1:0.8; 1:0.9; 1:1; 1:2; 1:3; 1:4; 1:5; 1:6; 1:7; 1:8; 1:9; 1:10; 1:11; 1:12; 1:13; 1:14; 1:15; 1:16; 1:17; 1:18; 1:19; 1:20; 1:21; 1:22; 1:23; 1:24; 1:25; 1:26; 1:27; 1:28; 1:29; 1:30; 1:31; 1:32; 1:33; 1:34; 1:35; 1:36; 1:37; 1:38; 1:39; 1:40: 1:41; 1:42; 1:43; 1:44; 1:45; 1:46; 1:47; 1:48; 1:49; 1:50 or about 1:100. These values can also be used to define a range, such as from about 1:0.0006 to about 1:48.

The molar ratio of vitamin B12 to choline can be about, more than about, or less than about 1:5; 1:10; 1:14; 1:15; 1:20; 1:50; 1:100; 1:200; 1:500; 1:1000; 1:2000; 1:5000; 1:10000; 1:20000; 1:50000, 1:1,000,000; 1:2,000,000; 1:5,000,000; 1:10,000,000; 1:20,000,000; 1:50,000,000; 1:100,000,000; 1:180,000,000; 1:200,000,000 or about 1:250,000,000. These values can also be used to define a range, such as from about 1:14 to about 1:200,000,000.

It is known that choline salts are inherently hygroscopic. They absorb water from the air and within a short time, e.g., a few hours, can form agglomerates or hard blocks. The uptake of water by choline salts can produce an opal like crystal which is difficult to breakup and, when in its complete form, is also difficult to hydrate. In some embodiments, the choline, choline salt, and compositions of the present disclosure, can be combined with a sugar alcohol, an alginate or derivatives thereof, or combinations thereof to reduce the hydroscopic nature of choline, see U.S. patent application Ser. No. 15/171,816, the content of which is incorporated herein by reference in its entirety.

The choline in the composition can be provided free of organic solvents. The choline in the composition can contain no detectable amounts of an organic solvent or less than about 0.1, 1 or about 10 ppm of organic solvent. In some embodiments, the choline in the composition can contain an organic solvent, or trace amounts of an organic solvent.

As provided above, choline can be provided, whole or in part, in other forms. The percentage of choline in the composition in the form of one or more phosphatidylcholines, lysophosphatidylcholines or combinations thereof can be about, more than about, or less than about, 1, 5, 10, 15, 20, 25, 30, 35, 40, 45, 50, 55, 60, 65, 70, 75, 80, 85, 90, 95, 99 or about 100%. These values can be used to define a range, such as about 20 to about 80%.

The present disclosure can also include a formulation, such as a capsule, for holding one or more substances in hermetic isolation. The formulation can include at least two separate chambers or sections that separate and segregate at least two of the components such as a powder, e.g., choline salt, and an oil, e.g., an essential fatty acid. See U.S. Patent Publication Nos. 20130186561, 20130233467, and 20140302133, the content of each of which is incorporated herein by reference in their entirety. The formulation can reduce or prevent the transfer of water, air, or other substances across one or more of the chambers. For example, the formulation can reduce or prevent a choline salt contained in the formulation from absorbing water from other components within the capsule, from outside the formulation, or both.

The capsule can be in the form of a digestible hard gelatin capsule for holding a substance such as a choline salt and/or an omega-3 oil. The capsule can contain one or more of the substances in hermetic isolation. Hermetic isolation can include both air-tight and liquid-tight chambers. Liquid samples can be prevented from leaking from a chamber. Substances susceptible to degradation by oxidation when exposed to oxygen present in the atmosphere can be protected.

The capsule can include, for example, a first capsule portion in the form of a capsule body and a second capsule portion in the form of a diaphragm. The capsule body can be made of a flexible gelatinous material and can be in the form of hollow cylindrical tubular body which defines a closed end and an opposed open end and which has a predetermined length dimension defined between the ends thereof. The diaphragm can be a flexible gelatinous material having a hollow cylindrical tubular body which defines a closed end and an opposed open end and which has a predetermined length dimension defined between the ends thereof, which is shorter than the length dimension of the capsule body. The diaphragm can be configured to seal off the body and provide a first compartment to hold a first substance. A cap can be applied to the body as is conventional in capsules. The space between the inner portion of the cap and the diaphragm can define a second compartment for holding a second substance. The contents of each compartment may be wet or dry (e.g., powder or liquid.). The capsule can contain one or more diaphragms and contain two or more compartments, e.g., 2, 3, 4, 5, or more compartments.

### Other Embodiments

Oral administration is one route for administration of compositions in accordance with the disclosure. Administration may be via capsule or enteric coated tablets, or the like. In making the compositions of the present disclosure, the components may optionally be combined with an pharmaceutically acceptable excipient or carrier. The composition can be in the form of a tablet, pill, powder, lozenge, sachet, cachet, elixir, suspension, emulsion, solution, syrup, sprinkle, shake, beverage, gel or liquid.

In other embodiments, the composition can include about 10 to about 1500 mg of an essential fatty acid selected from the group consisting of EPA, DHA and ALA, about 10 to about 33,000 μg of a cobalamin binding protein, about 0.25 to about 9,000 μg of vitamin B12, about 10 to about 5000 mg of choline, a biologically compatible salt thereof, or a phospholipid bound form and intermediates or derivatives thereof, or combinations thereof. The composition can include a molar ratio of essential fatty acid to cobalamin binding protein is about 1:0.025 to about 1:0.00000005, of the cobalamin binding protein to the vitamin B12 or a synthetic form of vitamin B12 is about 1:25,000 to about 1:0.00025, of the vitamin B12 or a synthetic form of vitamin B12 to choline is about 1:14 to about 1:200,000,000, and of choline to the essential fatty acid is about 1:0.0005 to about 1:50.

In the various embodiments of the present disclosure, the cobalamin binding protein (e.g., Intrinsic Factor) can bind to existing vitamin B12 in the body, such as ingested via diet and present in the GI tract. The cobalamin binding protein can enhance the existing vitamin B12's receptor mediated absorption. Further, vitamin B12 is continuously cycled via the bile through the entero-biliary pathway. The cobalamin binding protein can bind to existing vitamin B12 via this pathway for absorption of the vitamin B12 by the receptors in the ileum.

Additional embodiments of the composition of the present disclosure are provided in the Table below. Each embodiment can include one or more of the four components listed, e.g., EFA(s), CBP, B12 and Choline. In particular embodiments, the composition contains both EFA(s) and CBP, but optionally includes one or both of the B12 and choline. The amount of each component can also vary depending on the individual range selected. For example, one embodiment can contain EFA(s) and CBP according to the individual ranges selected from Nos. 5 and 29 (i.e., 300-340 mg EFA(s) and 180-220 μg cobalamin binding protein). In another example, an embodiment can contain all four components according to the individual ranges selected from Nos. 5, 29, 55 and 65 (i.e., 300-340 mg EFA(s), 180-220 μg cobalamin binding protein, 5.0-5.4 μg vitamin B12 and 50-70 mg choline). The essential fatty acids, choline or both can be present in free form, a biologically compatible salt thereof, or a phospholipid bound form and intermediates or derivatives thereof. The essential fatty acids can be present in any ratio of individual essential fatty acids, e.g., DHA, EPA and ALA. The vitamin B12 can be present as vitamin B12, a synthetic form of vitamin B12, or combinations thereof. The cobalamin binding protein can be present as provided herein.

| No. | EFA(s) | |
|---|---|---|
| 1 | 220-260 | mg |
| 2 | 240-280 | mg |
| 3 | 260-300 | mg |
| 4 | 280-320 | mg |
| 5 | 300-340 | mg |
| 6 | 320-360 | mg |
| 7 | 340-380 | mg |
| 8 | 360-400 | mg |
| 9 | 380-420 | mg |
| 10 | 400-440 | mg |
| 11 | 420-460 | mg |
| 12 | 440-480 | mg |
| 13 | 460-500 | mg |
| 14 | 480-520 | mg |
| 15 | 500-540 | mg |
| 16 | 520-560 | mg |
| 17 | 540-580 | mg |
| 18 | 560-600 | mg |
| 19 | 580-620 | mg |
| 20 | 600-640 | mg |
| No. | CBP | |
| 21 | 20-60 | μg |
| 22 | 40-80 | μg |
| 23 | 60-100 | μg |
| 24 | 80-120 | μg |
| 25 | 100-140 | μg |
| 26 | 120-160 | μg |
| 27 | 140-180 | μg |
| 28 | 160-200 | μg |
| 29 | 180-220 | μg |
| 30 | 200-240 | μg |
| 31 | 220-260 | μg |
| 32 | 240-280 | μg |
| 33 | 260-300 | μg |
| 34 | 280-320 | μg |
| 35 | 300-340 | μg |
| 36 | 320-360 | μg |
| 37 | 340-380 | μg |
| 38 | 360-400 | μg |
| 39 | 380-420 | μg |
| 40 | 400-440 | μg |
| No. | Vit B12 | |
| 41 | 2.2-2.6 | μg |
| 42 | 2.4-2.8 | μg |
| 43 | 2.6-3.0 | μg |
| 44 | 2.8-3.2 | μg |
| 45 | 3.0-3.4 | μg |
| 46 | 3.2-3.6 | μg |
| 47 | 3.4-3.8 | μg |
| 48 | 3.6-4.0 | μg |
| 49 | 3.8-4.2 | μg |
| 50 | 4.0-4.4 | μg |
| 51 | 4.2-4.6 | μg |
| 52 | 4.4-4.8 | μg |
| 53 | 4.6-5.0 | μg |
| 54 | 4.8-5.2 | μg |
| 55 | 5.0-5.4 | μg |
| 56 | 5.2-5.6 | μg |
| 57 | 5.4-5.8 | μg |
| 58 | 5.6-6.0 | μg |
| 59 | 5.8-6.2 | μg |
| 60 | 6.0-6.4 | μg |
| No. | Choline | |
| 61 | 10-30 | mg |
| 62 | 20-40 | mg |
| 63 | 30-50 | mg |
| 64 | 40-60 | mg |
| 65 | 50-70 | mg |
| 66 | 60-80 | mg |
| 67 | 70-90 | mg |
| 68 | 80-100 | mg |
| 69 | 90-110 | mg |
| 70 | 100-120 | mg |
| 71 | 110-130 | mg |
| 72 | 120-140 | mg |
| 73 | 130-150 | mg |
| 74 | 140-160 | mg |
| 75 | 150-170 | mg |
| 76 | 160-180 | mg |
| 77 | 170-190 | mg |
| 78 | 180-200 | mg |
| 79 | 190-210 | mg |
| 80 | 200-220 | mg |

The present disclosure also relates to a method of providing supplemental nutrition to a patient including administering to the patient of the composition of the present disclosure.

In one embodiment, the method can be optimizing the absorption of an essential fatty acid, protein, vitamins, choline, or combinations thereof by a patient including administering to the patient a composition of the present disclosure as described herein. As used herein the term optimizing can include increasing or decreasing a value by about 5%, 10, 20, 30, 40, 50, 60, 70, 80, 90, 100, 200, 300, 400 or about 500% in the direction of or approaching the value of a normal biological process. These values can also define a range, such as about 20 to about 50%. For example, the administration of the various compositions of the present disclosure, such as those containing cobalamin binding protein (e.g., IF), can enhance or increase the bioavailability of vitamin B12 by about, or more than about 5%, 10, 20, 30, 40, 50, 60, 70, 80, 90 or about 100% compared to natural biological process of the patient or compared to the patient not being administered such a composition (e.g., protein). These values can be used to define a range. Similarly, and in part due to the enhancement or increase of the bioavailability of vitamin B12, the administration of the various compositions of the present disclosure can preserve the amount of biological choline by 5%, 10, 20, 30, 40, 50, 60, 70, 80, 90 or about 100% compared to natural biological process of the patient or compared to the patient not being administered such a composition. These values can be used to define a range.

In one embodiment, the effectiveness of the compositions can be assessed using methylmalonic acid (MMA) as a marker for vitamin B12 deficiency. MMA is a dicarboxylic acid that functions as a metabolic substrate for the production of succinyl-CoA, which then enters the Krebb's Cycle for energy production. The conversion of methyl malonyl-CoA to succinyl-CoA requires adenosyl cobalamin as a co-factor and hence, when there is a cellular deficiency of vitamin B12, there is an upstream accumulation of MMA which can then build up in the blood and be excreted in the urine. It begins to build up shortly after a vitamin B12 deficiency begins and is one of the earliest and most sensitive and specific detectable markers of vitamin B12 deficiency. Any of the known standard MMA tests can be used to evaluate the effectiveness of the compositions of the present disclosure.

In one embodiment, the method can include optimizing the transport of an essential fatty acid, protein, vitamin, choline, or combinations thereof by administering to a patient a composition of the present disclosure as described herein. In particular, the method can optimize the transport of an essential fatty acid, such as DHA. DHA transport can be improved by administering or providing sufficient Intrinsic Factor to improve B12 transport, which can have a choline metabolic ‘saving’ effect. As such, when choline is administered or provided it is more metabolically available because adequate B12 is available. As a result, the metabolic availability of choline can enhance DHA transport.

Similarly, in other embodiments, the method can include optimizing the cellular uptake of an essential fatty acid, vitamin, choline, or combinations thereof by administering to a patient a composition of the present disclosure as described herein. The method can include optimizing the efficiency of an essential fatty acid, protein, vitamin, choline, or combinations thereof by administering to a patient a composition of the present disclosure as described herein.

In one embodiment, the method can be providing prenatal and postnatal nutrition to a patient including administering to the patient a composition of the present disclosure as described herein. A healthy lifestyle is vital during pregnancy. To ensure a healthy pregnancy mothers are encouraged to eat a proper diet, exercise and maintain an appropriate weight. The nutritional health of both the mother and baby are important before pregnancy, during pregnancy and after pregnancy. To function properly, the mother's reproductive system requires proper nutrients, vitamins and minerals, etc. To develop properly, the baby also requires proper nutrients, vitamins and minerals, etc.

A lack of proper nutrients during pregnancy can cause deficiencies which may negatively affect both mother and baby. Nutrition deficient mothers may lack the nutritional stores required to support embryo and fetus growth. Maternal malnutrition can adversely affect the division, replication and differentiation of cells in the embryo, impairing its development. Impaired embryo development in turn adversely effects the development of the fetus in the later stages of pregnancy. Maternal malnutrition also increases the risk of the baby being born at a low-birth weight. Low birth weight is in turn associated with a range of adverse outcomes in childhood and later in life. Maternal malnutrition can have a lifelong effect which predisposes the baby to chronic health conditions later in life, including neurodevelopmental, neurodegenerative and neuropsychiatric conditions.

The present disclosure relates to pharmaceutical compositions useful as a preconception, prenatal or peripartum supplement. The pharmaceutical compositions of the present disclosure can be used to treat or prevent the negative outcomes associated with nutritional deficiencies during pregnancy. The patient can be mother, pre- and post-pregnancy. The patient can be a baby, or offspring. The composition, or nutrition contained therein, can reduce the occurrence of a neural tube defect, low birth weight, premature birth, an allergic disease, a neurodevelopmental defect, a neurodegenerative defect, a neuropsychiatric defect, pervasive development disorders, autism, ADD, ADHD, schizophrenia, bipolar depression, Alzheimer's disease, Parkinson's disease, Huntington's disease, or combinations thereof. Treating with a composition of the present disclosure, or the nutrition contained therein, can reduce the occurrence of any one of these conditions, in a patient or in a group or class of patients, by about, or more than about 5%, 10, 20, 30, 40, 50, 60, 70, 80, 90, 100, 150 or about 200% compared to non-treatment or treating with a composition not containing all or some of the nutrients.

The composition, or nutrition contained therein, can reduce improve cognitive ability, a neurodevelopmental condition, a neurodegenerative condition, a neuropsychiatric condition, brain development, ocular health or combinations thereof. Treating with a composition of the present disclosure, or the nutrition contained therein, can improve any one of these conditions, in a patient or in a group or class of patients, by about, or more than about, 5%, 10, 20, 30, 40, 50, 60, 70, 80, 90, 100, 150 or about 200% compared to non-treatment or treating with a composition not containing all or some of the nutrients.

In some embodiments, the present disclosure relates to a method for preventing a complication of pregnancy, the method including administering to a subject in need thereof an effective amount of a combination including essential fatty acids, a cobalamin binding protein, choline, vitamin B12, or combinations thereof. In some embodiments, the majority of essential fatty acids, e.g., 50%+, is DHA or DHA containing compounds or components, or EPA or EPA containing compounds or components, or both. The essential fatty acids, choline, both, or portions of both can be in the form of one or more physiologically acceptable salts, phospholipid bound forms, intermediates or derivatives thereof.

The present disclosure also relates to a method for preventing a complication of pregnancy and/or improving the outcomes in the offspring, the method including administering to a subject in need thereof an effective amount of a combination including essential fatty acids, a cobalamin binding protein, choline, vitamin B12 or a synthetic form of vitamin B12, or combinations thereof. In some embodiments, the majority of essential fatty acids, e.g., 50%+, is DHA. The essential fatty acids, choline, both, or portions of both can be in the form of one or more physiologically acceptable salts, phospholipid bound forms, intermediates or derivatives thereof. The complication prevented or improved can be neural tube defects, low birth weight, prematurity, or combinations thereof. The improvement in the baby or offspring can include a reduction in allergic disease, improvement in cognitive or neurodevelopmental parameters, brain health, ocular health, or combinations thereof.

The present disclosure also relates to a method for preventing and/or improving neurodevelopmental, neurodegenerative and neuropsychiatric conditions the method including administering to a subject in need thereof an effective amount of a combination including essential fatty acids, a cobalamin binding protein, choline, vitamin B12 or a synthetic form of vitamin B12, or combinations thereof. In some embodiments, the majority of essential fatty acids, e.g., 50%+, is DHA or DHA containing compounds or components, or EPA or EPA containing compounds or components, or both. The essential fatty acids, choline, both, or portions of both can be in the form of one or more physiologically acceptable salts, phospholipid bound forms, intermediates or derivatives thereof. The conditions prevented or improved can include, but are not limited to, central nervous system disorders, such as pervasive development disorder, autism, ADD, ADHD, schizophrenia, bipolar disorder, bipolar depression, depression, Alzheimer's disease, Parkinson's disease, Huntington's disease, catalepsy, epilepsy, seizures, encephalitis, meningitis, migraine, tropical spastic paraparesis, arachnoid cysts, locked-in syndrome, Tourette's, multiple sclerosis and addiction. The conditions prevented or improved can also include, but are not limited to, inflammatory disorders, such as ankylosing spondylitis, arthritis, osteoarthritis, rheumatoid arthritis (RA), psoriatic arthritis, asthma, atherosclerosis, Crohn's disease, colitis, dermatitis, diverticulitis, fibromyalgia, hepatitis, non-alcoholic fatty liver disease (NAFLD), non-alcoholic steatohepatitis (NASH), metabolic syndrome, irritable bowel syndrome (IBS), systemic lupus erythematous (SLE), nephritis, ulcerative colitis and cardiovascular disease.

The method can include dosing the patient daily, b.i.d., t.i.d, or q.i.d. using one or more of the compositions described herein.

The disclosures of all cited references including publications, patents, and patent applications are expressly incorporated herein by reference in their entirety.

When an amount, concentration, or other value or parameter is given as either a range, preferred range, or a list of upper preferable values and lower preferable values, upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

The present invention is further defined in the following Examples. It should be understood that these Examples, while indicating preferred embodiments of the invention, are given by way of illustration only.

### EXAMPLES

### Example 1

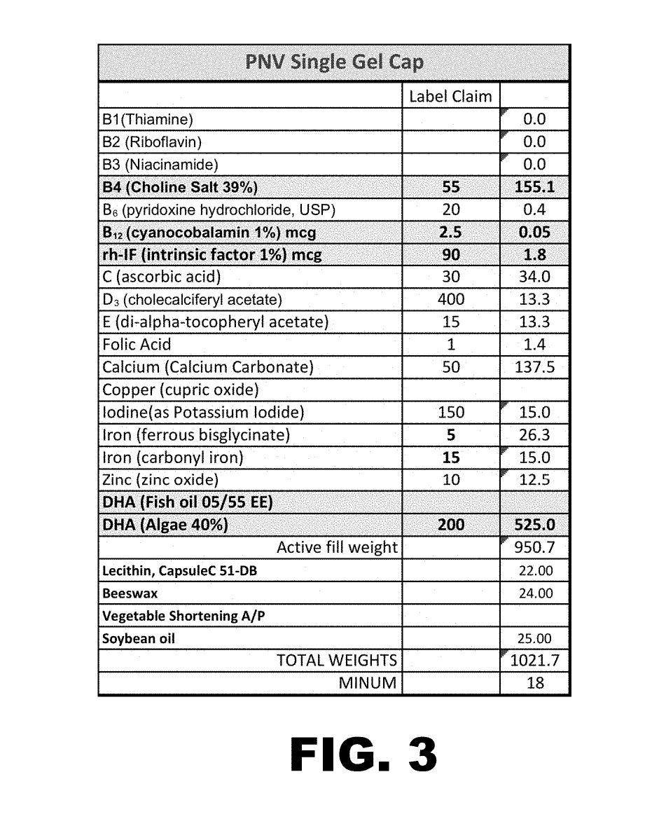

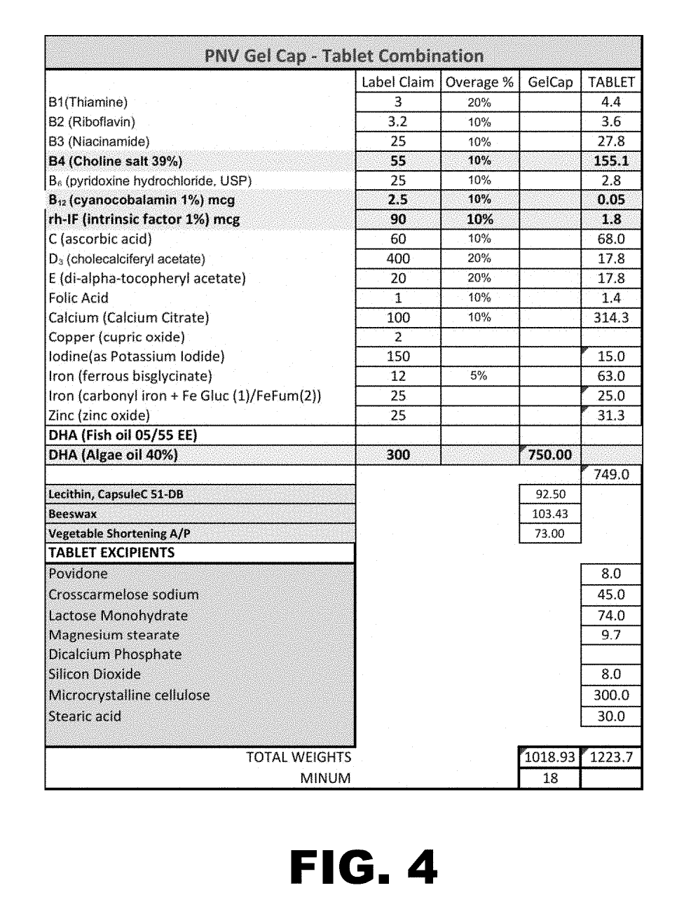

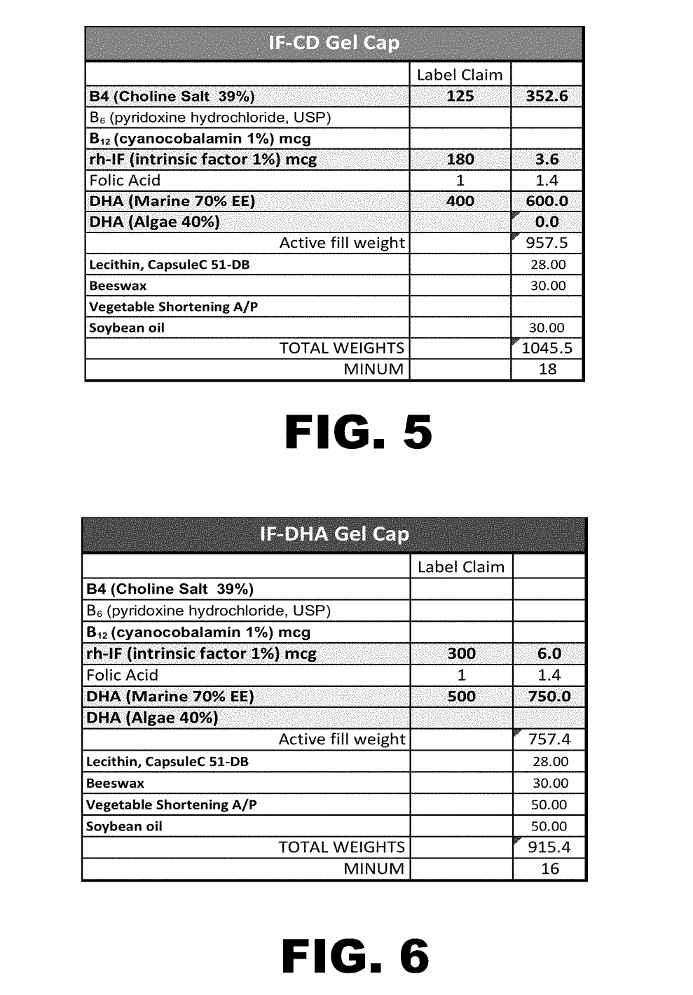

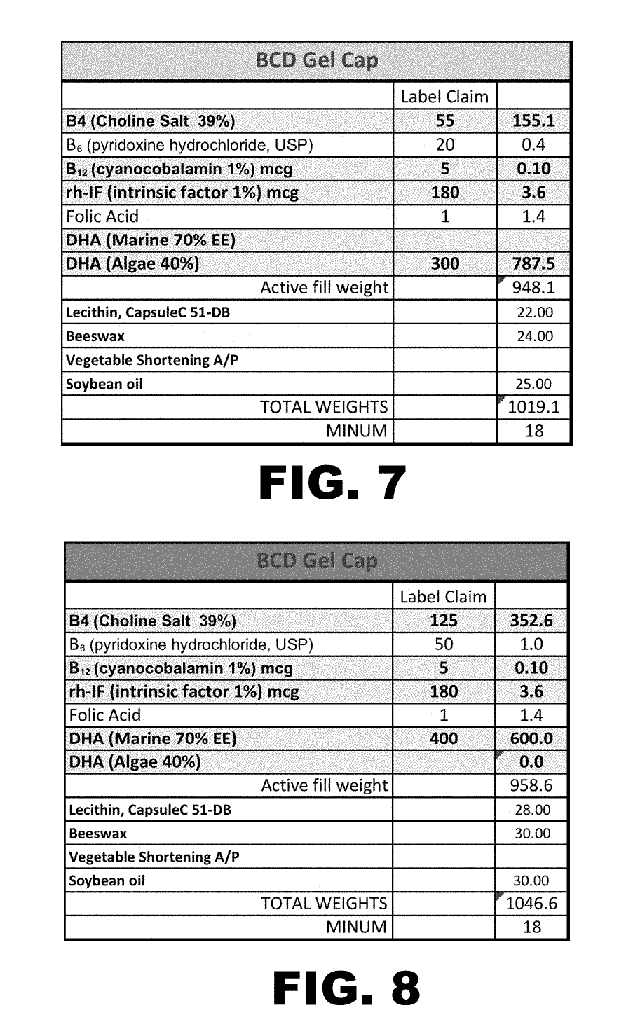

A pharmaceutical supplement is prepared containing 100 mg of choline tartrate, 400 mg of DHA, 4 μg of vitamin B12, 200 μg of Intrinsic Factor, and other additional ingredients in a gelatin capsule. FIGS. 3-8 show exemplary compositions of similar pharmaceutical supplement contemplated with the present disclosure.

Two groups of ten pregnant mothers are monitored for blood levels of choline, essential fatty acids EPA, DHA and ALA, and phosphatidylcholines during pregnancy and post-pregnancy. Prior to treating with the supplement, baseline blood levels are obtained from both groups. Starting at the beginning of the third trimester one group is treated daily with the supplement and the other is given a placebo. The daily supplement is orally administered to each mother in the selected group. For six months (last trimester of pregnancy and the first 90 days postpartum), the blood levels of choline, essential fatty acids EPA, DHA and ALA, and phosphatidylcholines in each mother from both groups are tested before and after administration of the supplement.

The pregnant mothers treated with the supplement exhibited greater levels of choline, essential fatty acids, in particular DHA, and phosphatidylcholines. Choline, DHA and phosphatidylcholines are individually increased in the treated mothers by about 5%, 10, 20, 30, 40, 50, 60, 70, 80, 90 or about 100% compared to the baseline levels and to the other untreated mothers. These values can be used to define a range, such as about 10 to about 50%.

### Example 2

Other pharmaceutical supplements are prepared. A pharmaceutical supplement is prepared containing about 55 mg of choline salt, about 300 mg of an essential fatty acid, about 5 μg of vitamin B12, and about 185.5 μg of Intrinsic Factor, in addition to other ingredients in a gelatin capsule.

Another pharmaceutical supplement is prepared containing about 10 mg of choline salt, about 1500 mg of an essential fatty acid, about 8880 μg of vitamin B12, and about 329,448 μg of Intrinsic Factor, in addition to other ingredients in a pharmaceutically acceptable dosage form.

Another pharmaceutical supplement is prepared containing about 10 mg of choline salt, about 1500 mg of an essential fatty acid, about 8880 μg of vitamin B12, and about 10 μg of Intrinsic Factor, in addition to other ingredients in a pharmaceutically acceptable dosage form.

Another pharmaceutical supplement is prepared containing about 3500 mg of choline salt, about 10 mg of an essential fatty acid, about 0.25 μg of vitamin B12, and about 33,000 μg of Intrinsic Factor, in addition to other ingredients in a pharmaceutically acceptable dosage form.

While this disclosure has been particularly shown and described with reference to example embodiments thereof, it will be understood by those skilled in the art that various changes in form and details may be made therein without departing from the scope of the invention encompassed by the appended claims.

We claim:

1. A composition comprising:
about 10 to about 1500 mg of an essential fatty acid selected from the group consisting of EPA, DHA and ALA; and
about 10 to about 33,000 μg of a cobalamin binding protein.

2. The composition of claim 1, wherein the essential fatty acid is DHA.

3. The composition of claim 2, wherein the source of DHA is animal, fish, plant, algae or microorganism produced.

4. The composition of claim 2, wherein the source of DHA is a phosphatidylcholine containing DHA, a lysophosphatidylcholine containing DHA, or combination thereof.

5. The composition of claim 1, wherein the cobalamin binding protein is Intrinsic Factor.

6. The composition of claim 1, wherein the cobalamin binding protein is recombinant human Intrinsic Factor.

7. The composition of claim 1, wherein the molar ratio of essential fatty acid to cobalamin binding protein is about 1:0.025 to about 1:0.00000005.

8. The composition of claim 1, further comprising about 0.25 to about 9,000 μg of vitamin B12 or a synthetic form of vitamin B12.

9. The composition of claim 8, wherein the vitamin B12 or synthetic form of vitamin B12 is selected from the group consisting of cyanocobalamin, methylcobalamin, hydroxocobalamin and adenosylcobalamin.

10. The composition of claim 8, wherein the molar ratio of the cobalamin binding protein to the vitamin B12 or a synthetic form of vitamin B12 is about 1:25,000 to about 1:0.00025.

11. The composition of claim 8, further comprising about 10 to about 3500 mg of choline, a biologically compatible salt thereof, or a phospholipid bound form and intermediates or derivatives thereof.

12. The composition of claim 11, wherein the molar ratio of choline to the essential fatty acid is about 1:0.0005 to about 1:50.

13. The composition of claim 11, wherein the molar ratio of the vitamin B12 or a synthetic form of vitamin B12 to choline is about 1:14 to about 1:200,000,000.

14. The composition of claim 11, wherein the choline is in the form of a salt.

15. The composition of claim 11, wherein the choline is in the form of choline chloride, choline bitartrate, choline hydroxide, choline citrate and choline carbonate or combinations thereof.

16. The composition of claim 11,
wherein the molar ratio of essential fatty acid to cobalamin binding protein is about 1:0.025 to about 1:0.00000005,
wherein the molar ratio of the cobalamin binding protein to the vitamin B12 or a synthetic form of vitamin B12 is about 1:25,000 to about 1:0.00025,
wherein the molar ratio of the vitamin B12 or a synthetic form of vitamin B12 to choline is about 1:14 to about 1:200,000,000, and
wherein the molar ratio of choline to the essential fatty acid is about 1:0.0005 to about 1:50.

17. A method of providing supplemental nutrition to a mammal comprising administering to the mammal the composition of claim 1.

18. The method of claim 17, wherein the nutrition reduces the occurrence of a neural tube defect, low birth weight, premature birth, an allergic disease, a neurodevelopmental defect, a neurodegenerative defect, a neuropsychiatric defect, pervasive development disorder, autism, ADD, ADHD, schizophrenia, bipolar depression, Alzheimer's disease, Parkinson's disease, Huntington's disease, Non-Alcoholic Fatty Liver Disease, Non-Alcoholic Steatohepatitis (NASH), Metabolic Syndrome, cardiovascular disease or combinations thereof.

19. The method of claim 17, wherein the nutrition improves cognitive ability, a neurodevelopmental condition, neurodegenerative condition, a neuropsychiatric condition, brain development and ocular health.
